(19) United States
(12) Patent Application Publication
(10) Pub. No.: US 2025/0000718 A1
(43) Pub. Date: Jan. 2, 2025

(54) INTELLIGENT ABSORBENT ARTICLE BASED ON SENSING FILM AND BUILT-IN POCKET AND RELATED DEVICE

(71) Applicants: Qianhai Innovation & Entrepreneur Hub, Shenzhen, Guangdong Province (CN); Sanwa Corporation, Tokyo (JP)

(72) Inventors: Sun Hoi WONG, Hong Kong (HK); Zhen CHEN, Hong Kong (HK); Fei XU, Hong Kong (HK)

(21) Appl. No.: 18/697,477

(22) PCT Filed: May 6, 2022
(86) PCT No.: PCT/CN2022/091190

§ 371 Date: Mar. 31, 2024

(30) Foreign Application Priority Data
Aug. 27, 2021 (CN) 202110992486.1

(51) Int. Cl. A61F 13/496; A61F 13/42

(52) U.S. Cl. CPC A61F 13/496; A61F 2013/422

(57) ABSTRACT

A smart absorbent article (10) based on a capacitive sensing film (40) and a built-in pocket (26), a system device implementing monitoring of the wetness state of the smart absorbent article (10), and two types of capacitive sensing film (40) including the built-in pocket (26) and suitable for producing the smart absorbent article (10). The smart absorbent article (10) includes a disposable absorbent article, a capacitive sensing film (40), a built-in pocket (26), and a short circuit protection mechanism. The disposable absorbent article includes a topsheet (11), an absorption layer, and a backsheet (15). The capacitive sensing film (40) includes a contact surface, a sensing surface (62), a dielectric layer, and at least two sensing lines (21,22); when the sensing surface (62) is wetted by liquid containing electrolytes, the sensing lines (21,22) can achieve a wetness detection function through the dielectric layer by means of electrolytic capacitance.

### BACKGROUND

### Priority Claim

This disclosure claims the priority of patent application No. 202110992486.1 filed with the Chinese Patent Office on Aug. 27, 2021, entitled “A smart absorbent article with sensing lines and an invisible pocket”, all of which are incorporated by reference in the present disclosure.

### TECHNICAL FIELD

The present disclosure relates to an absorbent article, and in particular, to a smart disposable absorbent article and a related device based on a capacitive sensing film and a built-in pocket.

### RELATED ART

Disposable absorbent articles include diapers, nappies, sanitary napkins, urine pads and other sanitary products, which are disposable care products and should be replaced as needed when in use. If changed too early/too often, it will increase workload and cause waste; If changed too late, it is easy to cause leakage, and the excrement stimulates the skin for a long time and is easy to cause skin diseases. Therefore, people look forward to a smart disposable absorbent article that can detect moisture/wetness in real time and prompt to change the diaper at the right time, which has a positive significance for scientific use of the products.

In terms of prior art, China Patent publication No. CN107174408B discloses an absorbent article and related method, which realizes the accurate, reliable, and quantified wetness detection function based on electrolytic capacitance by setting two sensing lines (flexible electrodes) on the outer surface of the backsheet of the diaper, which is one of the most representative solutions among the prior art. When it is used, a sensor is attached to the outer surface of the diaper first, and then open the sensor flap, align the probe with the sensing lines on the diaper, and close it, so that the probe can get connected with the sensing lines and detect the wetness state of the diaper in real time.

The shortcomings of the above prior art mainly include: (1) the sensing lines are printed on the outer surface of the backsheet of the diaper with carbon ink, which will produce two black lines and affect the appearance of the diaper. (2) the sensing lines set on the outer surface of the backsheet of the diaper is easy to be affected by external moisture, such as touching the diaper with a wet hand will cause false alarm. (3) the sensor is hard to secure on the outside of diaper. (4) the sensor probe must be tied through the outer non-woven fabric of the diaper to connect with the sensing lines, which is easily lead to poor contact. (5) the alignment between the sensor and the sensing lines depends on the naked eye and experience, which is easy to cause misalignment and connection failure.

In order to solve the above problems, China Patent publication No. CN1226855A discloses a wetness detection device for diapers and other diapers with such a device. Its sensing lines are arranged inside the diaper and a pocket is arranged on the outer surface of the diaper, and the elasticity of the pocket itself holds the sensor in place, and the sensor located on the outside of the diaper is then capacitively coupled to the sensing lines located inside the diaper for wetness detection.

The main shortcomings of this prior art include: (1) the sensor and the sensing lines are not face-to-face contact; it is difficult to achieve a reliable electrical connection. (2) the pocket and the sensing lines are located on both sides of the diaper, easy to cause dislocation. (3) the sensing lines detect wetness by direct contact with urine, which is a traditional resistive type of wet alarm system and cannot achieve accurate and reliable wetness level detection based on electrolytic capacitance. (4) the pocket set outside the diaper cannot use the waist elasticity generated by the diaper in the wearing state to fix the sensor and cannot make the electrical connection between the sensor contacts and the sensing lines more reliable.

The shortcomings of the prior art need to be solved by new technical solutions.

### SUMMARY

The technical problems to be solved by the present disclosure are mainly the deficiencies in the prior art mentioned in the background. In order to solve these problems, the measures taken by the present disclosure mainly include:

1. The sensing lines will be set inside the diaper to solve the problem that the sensing lines affects the appearance of the diaper and is affected by external moisture.

2. Set up a short-circuit protection mechanism in the diaper, so that the sensing lines are protected from the influence of moisture inside the diaper, and achieve accurate, reliable, and quantifiable wetness detection function based on electrolytic capacitance.

3. Set a pocket inside the diaper to solve the problem of fixing the sensor.

4. Make the sensor contacts face to face with the sensing lines to solve the reliability of the electrical connection.

5. Take measures to make the built-in pocket can be automatically centered and aligned with the sensing lines to ensure that the sensor will not be misaligned when inserted into the pocket.

6. Provide a sensing film with a built-in pocket as a raw material for the production of smart diapers, which brings convenience to the mass production of products.

7. Provide a solution that can use the elastic force generated on the waist when the diaper is worn to fix the sensor and make the electrical connection between the sensor and the sensing lines more reliable.

In order to solve the above technical problems, in the first aspect, the present disclosure provides a smart absorbent article based on a capacitive sensing film and a built-in pocket, including a disposable absorbent article, a capacitive sensing film, a built-in pocket and a short-circuit protection mechanism, the disposable absorbent article includes a topsheet, an absorbent layer and a backsheet, and the sensing film includes a contact surface, a sensing surface, a dielectric layer and at least two sensing lines, the outer surface of the dielectric layer constitutes the sensing surface, the dielectric layer separates and insulates the sensing lines and the sensing surface, when the sensing surface is soaked by a liquid containing electrolyte, the sensing lines can realize the wetness detection function of the sensing surface through the dielectric layer by means of electrolytic capacitance, the built-in pocket is adapted to insert a sensor, the contact surface facilitates face-to-face contact and electrical connection between the sensor and the sensing lines, the short-circuit protection mechanism is adapted to protect the sensing lines from moisture inside and outside the disposable absorbent article.

Wherein at least part of the sensing lines is located within the contact surface and includes exposed part, the short-circuit protection mechanism includes a waterproof covering layer which covers the contact surface, and a separable part between the contact surface and the waterproof covering layer constitutes the built-in pocket, the sensing lines can realize the wetness detection function of the absorbent article through the dielectric layer by means of electrolytic capacitance.

Wherein the capacitive sensing film includes a single-sided sensing film, the dielectric layer includes a plastic film substrate, the sensing lines are arranged on one side of the plastic film substrate to form the contact surface, the other side of the plastic film substrate to form the sensing surface, the backsheet to form the waterproof covering layer, the contact surface is oriented towards the backsheet, a separable part is included between the contact surface and the backsheet and constitutes the built-in pocket, the absorbent layer is covered on the sensing surface, the topsheet is covered on the absorbent layer, the topsheet beyond the absorbent layer and the sensing surface part is bonded with the backsheet, the built-in pocket is thus fixed and aligned with the sensing film width; or

The capacitive sensing film includes a composite sensing film, the composite sensing film includes a plastic film substrate, the sensing lines are arranged on one side of the plastic film substrate, the composite sensing film also includes a contact area and an sensing area, the sensing area includes a waterproof protective layer and protects the sensing lines, the outer surface of the plastic film substrate is oriented towards the absorbent layer and constitutes the dielectric layer, the sensing lines on the contact area includes a bare part and constitutes the contact surface, the backsheet constitutes the waterproof covering layer and covers the contact surface, the absorbent layer covers the sensing surface, the topsheet covers the absorbent layer, and the topsheet covers the absorbent layer, the part of the topsheet beyond the absorbent layer and the sensing surface is bonded with the backsheet, and the built-in pocket is fixed and aligned with the width of the capacitive sensing film; or

The capacitive sensing film includes a composite sensing film, the composite sensing film includes a plastic film substrate, the sensing lines are arranged on one side of the plastic film substrate, the composite sensing film also includes a contact area and a sensing area, the sensing area includes a waterproof protective layer and protects the sensing lines, the sensing lines on the contact area includes a bare part and constitutes the contact surface, the waterproof protective layer is oriented towards the absorbent layer and constitutes the dielectric layer, the sensing film is located between the topsheet and the backsheet and is bonded with the topsheet, the waterproof covering layer and the backsheet, thereby fixing the built-in pocket and making it aligned with the sensing film width; or

The capacitive sensing film includes a sensing film with a head-and-tail built-in pocket, the sensing film includes a plastic film substrate, the sensing lines are arranged on one side of the plastic film substrate, the waterproof covering layer is covered on the sensing lines, the waterproof covering layer and the plastic film substrate include a mutually bonded part in the middle section and constitute a sensing area, and include a mutually separable part at both ends of the head and tail and constitute the built-in pocket, the outer surface of the plastic film substrate or the waterproof covering layer is oriented towards the absorbent layer; or

The capacitive sensing film includes a sensing film with a through-type built-in pocket, the sensing film includes a plastic film substrate, the sensing lines are arranged on one side of the plastic film substrate and constitutes the contact surface, the other side of the plastic film substrate constitutes the sensing surface, the plastic film substrate constitutes the dielectric layer, the waterproof covering layer is covered on the contact surface, the edge of both sides of the waterproof covering layer and the plastic film substrate includes a part bonded to each other and constitutes a boundary preventing liquid penetration into, a hollow port is further included between the waterproof covering layer and the plastic film substrate and constitutes the through-type built-in pocket; or

The capacitive sensing film includes a composite backsheet type sensing film, the sensing lines are arranged on the outer surface of the backsheet to form the contact surface, the inner surface of the backsheet towards the absorbent layer to form the sensing surface, the backsheet to form the dielectric layer, the waterproof covering layer includes an integrated cut intact outer non-woven fabric, it covers the contact surface and is combined with the backsheet to form a composite backsheet, the outer non-woven fabric and the backsheet also include a mutually separable part between the outer non-woven fabric and the backsheet to form the built-in pocket; or

Including an outer non-woven fabric, the capacitive sensing film includes a composite backsheet type sensing film, the sensing lines are arranged on the outer surface of the backsheet to form the contact surface, the inner surface of the backsheet towards the absorbent layer to form the sensing surface, the backsheet to form the dielectric layer, the waterproof covering layer covers the sensing lines, the outer non-woven fabric covers the waterproof covering layer, the part that exceeds the waterproof covering layer in width is bonded with the backsheet to form a composite backsheet, thereby fixing the waterproof covering layer and the built-in pocket and making the built-in pocket consistent with the width of the waterproof covering layer.

Wherein the sandwich at the front edge of the disposable absorbent article includes an invisible opening, which leads to the built-in pocket for inserting a sensor to achieve the wetness detection function; or

The sandwich at the front edge of the disposable absorbent article and the back end edge of the disposable absorbent article includes an invisible opening, each leading to a built-in pocket, wherein the built-in pocket located at the front edge constitutes a functional pocket for inserting a sensor to achieve the wetness detection function, the built-in pocket located at the edge of the back end constitutes a redundant pocket to provide error redundancy for the positioning cut-off during the production of the smart absorbent article; or

The outer surface of the disposable absorbent article or the inner surface of the front edge of the disposable absorbent article includes an opening, which leads to the built-in pocket for inserting a sensor to achieve the wetness detection function.

Wherein the disposable absorbent article includes a front belly patch and a left and right side patch, and at least part of the built-in pocket is located within the ortho projection range of the front belly patch; when the disposable absorbent article is in the wearing state, the elastic force generated on the waist circumference can be converted into the force that attaches the built-in pocket to the front abdomen of the user, thereby pressing the sensor arranged in the built-in pocket and making the contact of the sensor with the sensing lines more reliable; when the disposable absorbent article is in a non-wearing state, the force attached to the front abdomen of the user automatically disappears and the sensor is easily removed.

Wherein the disposable absorbent article includes an elastic waist circumference, the built-in pocket is arranged on the elastic waist circumference, when the smart absorbent article is in a wearing state, the elasticity generated on the elastic waist circumference can be converted into the force of the built-in pocket attached to the user's body, the sensor arranged in the built-in pocket is thus compressed and the contact of the sensor with the sensing lines is made more reliable.

Wherein the disposable absorbent article includes a diaper, an insertion pad, a sanitary napkin, a maternal towel or an urine pad, the topsheet includes a hydrophilic non-woven fabric, the absorbent layer includes polymer absorbent material, the backsheet includes a breathable or impermeable PE film, the sensing lines include ink lines printed with conductive ink, metal lines formed by physical vapor deposition, aluminum foil or copper foil, the plastic film substrate include a PE, PP, CPP, BOPP or PET film, and the waterproof covering layer includes a plastic film, a waterproof paper or a water-repellent non-woven fabric, the waterproof protective layer includes a polymer waterproof coating or a polymer waterproof composite layer, and the electrolyte containing liquid includes urine, loose stool, sweat or blood containing salt.

Wherein when the disposable absorbent article is soaked by the liquid containing electrolytes, the sensing lines, the dielectric layer, and the liquid together to form an electrolytic capacitor, where the sensing lines constitute the electrodes of the electrolytic capacitor, the dielectric layer constitutes the dielectric medium of the electrolytic capacitor, the liquid constitutes the electrolyte of the electrolytic capacitor, the wetness level of the disposable absorbent article is proportional to the capacitance of the electrolytic capacitor, the capacitance is proportional to the area of the liquid corresponding to the sensing lines, is proportional to the dielectric constant of the dielectric layer, and is inversely proportional to the thickness of the dielectric layer.

In the second aspect, the present disclosure provides a capacitive sensing film including a head-and-tail built-in pocket for making the smart absorbent article, including a plastic film substrate, a waterproof covering layer and at least two sensing lines, the sensing lines are arranged on one side of the plastic film substrate, the waterproof covering layer covers the sensing lines, the waterproof covering layer and the plastic film substrate include a mutually bonded part in the middle part to form a protected sensing area for preventing liquid penetration into the area and causing a short circuit of the sensing lines, the waterproof covering layer and the plastic film substrate include a mutually separable part at both ends of the head and tail and constitute a contact area, wherein the contact area located at the front edge constitutes a functional pocket, the sensing lines are exposed in the functional pocket and constitutes a contact surface for face-to-face contact and electrical connection with the contact points of the sensor inserted into the pocket, and the contact area located at the back end edge constitutes a redundant pocket, which can provide error redundancy for the positioning cutting during the production of the smart absorbent article; and

The plastic film substrate or the waterproof covering layer constitutes the dielectric layer, the outer surface of which constitutes the sensing surface, the sensing lines can detect the wetness state of the sensing surface through the dielectric layer and by means of electrolytic capacitance, and the detection sensitivity is proportional to the dielectric constant of the dielectric layer and inversely proportional to the thickness of the dielectric layer.

Wherein the sensing lines include ink lines printed by conductive ink, metal lines formed by physical vapor deposition, aluminum foil or copper foil, and the plastic film substrate includes a PE, PP, CPP, BOPP or PET film, the waterproof covering layer includes a plastic film, a waterproof paper, a water-repellent non-woven fabric or a hot melt adhesive film.

In the third aspect, the present disclosure provides a capacitive sensing film including a through-type built-in pocket for making the smart absorbent article, including a plastic film substrate, a waterproof covering layer and at least two sensing lines, the sensing lines are arranged on one side of the plastic film substrate, the waterproof covering layer covers the sensing lines, both sides of the waterproof covering layer and the plastic film substrate include a part at the edge that is bonded to each other and thus generates a boundary that prevents liquid penetration and constitutes a short-circuit protection mechanism, the waterproof covering layer and the plastic film substrate includes a hollow part and constitutes a through-type built-in pocket, the sensing lines are exposed in the built-in pocket and constitutes the contact surface for face-to-face contact and electrical connection with the contacts of a sensor inserted into the built-in pocket; and

The plastic film substrate constitutes the dielectric layer, the outer surface of which constitutes the sensing surface, the sensing lines can detect the wet state of the sensing surface through the dielectric layer by means of electrolytic capacitance, and the detection sensitivity is proportional to the dielectric constant of the dielectric layer and inversely proportional to the thickness of the dielectric layer.

Wherein the sensing lines include ink lines printed by conductive ink, metal lines formed by physical vapor deposition, aluminum foil or copper foil, and the plastic film substrate includes a PE, PP, CPP, BOPP or PET film, the waterproof covering layer includes a plastic film, a waterproof paper, a water-repellent non-woven fabric or a hot melt adhesive film.

In the fourth aspect, the present disclosure provides system device for monitoring the wet state of a smart absorbent article, including a sensor and a smart absorbent article, wherein the sensor includes at least two contacts for contacting and electrically connecting the sensing lines in the built-in pocket, and the sensor also includes a capacitance detecting device for realizing the wetness detection function of the disposable absorbent article by means of electrolytic capacitance.

Wherein the sensor includes at least three contacts, two of which constitute an insertion detection device, when the sensor is inserted into the built-in pocket and the two contacts contact the same sensing line at the same time, the insertion detection device is triggered, and the sensor enters working state.

Wherein the sensor also includes a wireless transmitter and a wireless receiving device for sending and receiving wetness information of the smart absorbent article by wireless means, and the wireless receiving device includes an audible and visual alarm device, a smart phone, or a personal computer.

The present disclosure has the beneficial effect of solving the shortcomings of the prior art mentioned in the background, including: (1) the sensing lines are successfully arranged in the inside of the diaper, eliminating the influence of the color of the sensing lines on the appearance of the diaper. (2) the sensing lines are protected from short circuit and are no longer affected by moisture inside or outside the diaper. (3) the pocket set inside the diaper can more effectively contain and fix the sensor. (4) the sensor and the sensing lines to achieve face to face contact and make the connection more reliable. (5) the built-in pocket and sensing film are automatically positioned and aligned to avoid misalignment. (6) the sensing film can be used as a general production material, with a wider range of versatility and convenience. (7) the elastic force generated on the waist when the diaper is worn is cleverly used to fix the sensor and make the connection between the sensor and the sensing lines more reliable.

The present disclosure solution effectively improves the reliability and convenience of the product and creates favorable conditions for the development of the diaper industry and smart upgrade of diaper products.

### BRIEF DESCRIPTION OF THE DRAWINGS

In order to state the technical scheme of the embodiments of the present disclosure more clearly, the following is a brief introduction of the drawings required to be used in the embodiments. Obviously, the drawings described below are only some embodiments of the preset disclosure, and other drawings can be obtained according to these drawings for ordinary technicians in the field without creative effort.

FIG. 1 illustrates a schematic diagram of the appearance structure of a smart absorbent article when it is used together with a sensor according to an embodiment of the present disclosure.

FIG. 2A and FIG. 2B illustrate a three-dimensional structure diagram and a cross section structure diagram/equivalent circuit diagram of a single-sided sensing film suitable for the smart absorbent article according to an embodiment of the present disclosure.

FIG. 3A and FIG. 3B illustrate an A-A′ ‘cross section structure diagram and a B-B’ cross section structure diagram/equivalent circuit diagram of a smart absorbent article using the single-sided sensing film according to an embodiment of the present disclosure.

FIG. 4A and FIG. 4B illustrate two schematic diagrams of the appearance structure of a smart absorbent article using the single-sided sensing film when it is used together with a sensor according to an embodiment of the present disclosure.

FIG. 5A and FIG. 5B illustrate a three-dimensional structure diagram and a cross section structure diagram/equivalent circuit diagram of a composite sensing film suitable for a smart absorbent article according to an embodiment of the present disclosure.

FIG. 6A and FIG. 6B illustrate an A-A′ cross section structure diagram and a B-B′ cross section structure diagram/equivalent circuit diagram of a smart absorbent article using the composite sensing film according to an embodiment of the present disclosure.

FIG. 7 illustrates a schematic diagram of the appearance structure of a smart absorbent article using the composite sensing film when it is used together with a sensor according to an embodiment of the present disclosure.

FIG. 8A, FIG. 8B and FIG. 8C illustrate three schematic diagrams of C-C′ cross section structure of a smart absorbent article using the composite sensing film according to an embodiment of the present disclosure.

FIG. 9A and FIG. 9B illustrate an A-A′ cross section structure diagram and a B-″ cross section structure diagram/equivalent circuit diagram of a smart absorbent article using a composite backsheet type sensing film according to an embodiment of the present disclosure.

FIG. 10A and FIG. 10B illustrate a front structure diagram and a D-D′ cross section structure diagram of a capacitive sensing film including a head-and-tail built-in pocket suitable for used in a smart absorbent article according to an embodiment of the present disclosure.

FIG. 11A, FIG. 11B and FIG. 11C illustrate a front structure diagram, an E-E′ cross section structure diagram and a position relationship diagram of a capacitive sensing film with a through-type built-in pocket, which are suitable for the use of a smart absorbent article according to an embodiment of the present disclosure.

FIG. 12 illustrates a schematic diagram of the appearance structure of a smart absorbent article (with a built-in pocket on the elastic waist) according to an embodiment of the present disclosure.

FIG. 13 illustrates a schematic diagram of the appearance structure of a smart absorbent article according to an embodiment of the present disclosure, whose outer face includes an incision leading to a built-in pocket.

FIG. 14 and FIG. 15 illustrate schematic diagrams of appearance structure of sensing films suitable for use in a smart absorbent article having a curved sensing lines design and a plurality of sensing lines according to an embodiment of the present disclosure.

### DETAILED DESCRIPTION

The descriptions of the following embodiments refer to the attached drawings and serve as examples of specific embodiments in which the present disclosure can be implemented. The direction and position terms mentioned in the present disclosure, such as “up”, “down”, “front”, “back”, “left”, “right”, “inside”, “outside”, “top”, “bottom”, “side”, etc., only refer to the direction or position of the attached drawings. Therefore, the direction and position terms used are used to describe and understand the present disclosure, and not to limit the scope of protection of the present disclosure.

FIG. 1 is a schematic diagram of the appearance structure of a smart absorbent article when it is used together with a sensor in an embodiment of the present disclosure, which is almost the same in appearance as a traditional disposable absorbent article. Diapers are the most representative of disposable absorbent articles, and the embodiment of the present disclosure will mainly take diapers as an example to illustrate, and the relevant descriptions are also suitable for disposable absorbent articles such as pull-up pants, training pants, insertion pads, sanitary napkins, maternity napkins, urine pads, etc. In most cases, the diapers discussed in embodiments of the present disclosure have the same meaning as disposable absorbent articles.

The smart absorbent article 10 of the embodiment of the present disclosure is realized on the basis of a traditional diaper by adding some materials or devices that can sense (detect) wetness (moisture), which make the diaper smart, so it can be referred to as a smart diaper. Diapers usually include a backsheet (leakproof layer), an absorbent layer (sandwich), a topsheet (dry layer), an outer non-woven fabric (composite layer) and other components, the backsheet usually includes a breathable or non-breathable polyethylene film (PE), the absorbent layer usually includes polymer absorbent material (SAP) and wood pulp, the topsheet usually includes a soft and comfortable hydrophilic non-woven fabric. The outer layer of non-woven fabric is usually combined with the backsheet to form a composite backsheet, which can make the backsheet have a better feel and increase the strength of the backsheet.

The front 18 of the diaper can also be referred to as the front belly, front waist, or diaper head, while the back 19 of the diaper can be referred to as the rear, back waist, or diaper tail. A typical diaper may also include left and right stickers/adhesive strips/Velcro 16 and 17, which are located on the left and right sides of the front abdomen of the diaper respectively and are attached to the front belly patch/front waist patch 25 when in use, which pulls the waist of the diaper tight to prevent the diaper from slipping off.

In terms of wetness detection, the embodiment of the present disclosure is mainly realized by setting a capacitive sensing film (referred to as sensing film) material containing detection electrodes inside the diaper. The sensing film includes at least two detection electrodes/flexible electrodes/wetness sensing lines (collectively referred to as sensing lines). For example, the sensing film 40 in this embodiment includes a first sensing line 21 and a second sensing line 22. The sensing lines usually start at the front edge of the diaper and reach or approach the back edge of the diaper across the absorbent layer 12 area (crotch) of the diaper to achieve wetness detection of the entire diaper. The smart absorbent article of the embodiments of the present disclosure also includes a built-in pocket 26 below the front belly patch 25 to accommodate a sensor 30, and the sensing film/sensing lines is at least partially located within the pocket to contact and perform electrical connection with the sensor inserted into the pocket.

The sensing film 40 and the built-in pocket 26 can be arranged between the backsheet and the topsheet of the diaper or between the backsheet and the outer non-woven fabric. This embodiment uses the gap in the interlayer at the front edge of the diaper as an opening for the built-in pocket 26 to insert the sensor. The sensor 30 in this figure includes two contacts 31 and 32, which are in face-to-face contact with the first and second exposed sensing lines 21 and 22 in the built-in pocket and electrically connected and can detect the wetness by means of electrolytic capacitance, thus realizing the wetness detection function of the diaper.

The sensing film of the embodiments of the present disclosure is a flexible film including two outer surfaces, and the side in contact with the excrement/liquid to be measured is called the sensing surface. The sensing film can also be divided into contact area and sensing area, the sensing area is usually corresponding to the absorbent layer of the diaper and is mainly used to detect the wetness of the diaper, while the contact area is mainly used to contact the sensor contacts and implement electrical connection. The plane in contact with the sensor contacts is called the contact surface, and at least part of the contact surface is located within the built-in pocket 26. And at least part of the sensing lines is exposed on the contact surface in order to implement electrical connection with the contact of the sensor.

The sensing lines of the embodiments of the present disclosure is non-contact with the liquid to be measured, and the sensing lines will implement the wetness detection of the diaper through non-contact capacitive sensing (capacitive coupling). In order to prevent the liquid from penetrating into the sensing lines area, the embodiments of the present disclosure also include a short-circuit protection mechanism, which is to prevent the sensing lines from coming into contact with the liquid containing electrolytes to short-circuit and cause detection failure or lead to false triggering/false alarm. Because the sensing film is arranged inside the diaper, the diaper of the embodiment of the present disclosure is no different from the traditional diaper from the outside, and the sensing film/sensing line has almost no influence on the appearance of the diaper. In order to illustrate the relationship between the sensing film, sensing line, contact surface, sensing surface of the embodiment of the present disclosure and the topsheet, absorbent layer, backsheet and outer non-woven fabric of the diaper, an A-A′ and a B-B′ sections related to the embodiment shown in FIG. 1 are further explained in the following legends.

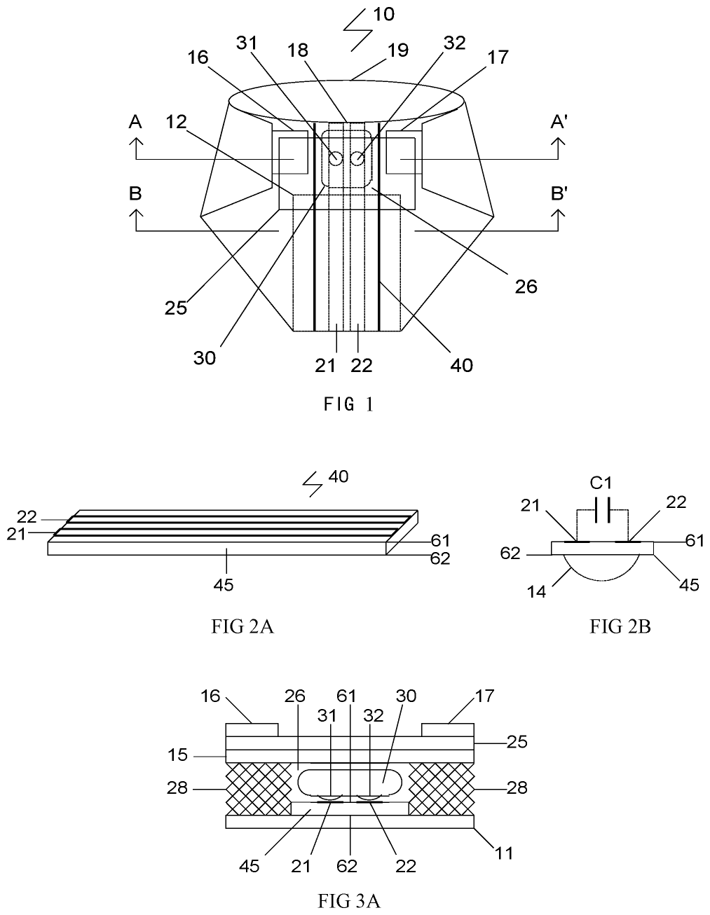

FIG. 2A and FIG. 2B are three-dimensional structure diagram and cross section structure diagram/equivalent circuit diagram of a single-sided sensing film suitable for a smart absorbent article of an embodiment of the present disclosure. The sensing film 40 of the present embodiment is a single-sided capacitive sensing film including a film substrate (the substrate) 45 and a first sensing line 21 and a second sensing line 22 arranged on one side of the substrate. The sensing lines will detect the wetness state on the other side of the substrate 45 by means of electrolytic capacitance.

The sensing lines in the embodiments of the present disclosure generally include ink lines (conductive film) printed with conductive ink (such as carbon paddle or silver paste), metal lines (metal film) of physical vapor deposition (such as vacuum evaporation aluminum) or aluminum foil, copper foil, etc. In this embodiment, the side of the substrate 45 with sensing lines is referred to as the contact surface 61, and the other side as the sensing surface 62, and the substrate 45 insulating the sensing lines and the sensing surface separated from each other as the dielectric layer. Commonly used substrates include PE (polyethylene) film, PP (polypropylene) film, CPP (casting polypropylene) film, BOPP (biaxial tensile polypropylene) film, PET (polyester) film and other plastic films, so it can be called plastic film substrate, its thickness is generally between 5 microns and 50 microns.

When a liquid containing electrolyte (such as urine containing salt, loose stool, sweat, menstrual blood, blood and other liquids, referred to as liquid) 14 accumulates on the sensing surface 62, the sensing lines 21 and 22 together with the liquid 14 and the plastic film substrate 45 form an electrolytic capacitor C1, in which the sensing lines 21 and 22 form the electrodes of C1. The plastic film substrate 45 constitutes the dielectric of C1, the liquid 14 constitutes the electrolyte of C1, the capacitance of C1 is inversely proportional to the thickness of the dielectric, is proportional to the dielectric constant, and is proportional to the area corresponding to the liquid and the electrodes (sensing lines), by reading the capacitance value of C1 can accurately know the quantitative wetness state of the sensing surface.

It should be noted that the electrolytic capacitance of the embodiments of the present disclosure is generated based on the conductivity of human excrement, which is essentially different from the dielectric capacitor mentioned by some prior art which treats urine as a non-conductive liquid with a high dielectric constant (e.g. 80). In fact, there is also some dielectric capacitance unrelated to electrolytic capacitor generated between the first and second sensing lines of the embodiments of the present disclosure, but this dielectric capacitance is much smaller than the electrolytic capacitance when the diaper is wet, so the embodiments of the present disclosure ignore the dielectric capacitance and only discuss the electrolytic capacitance generated by the electrolyte liquid.

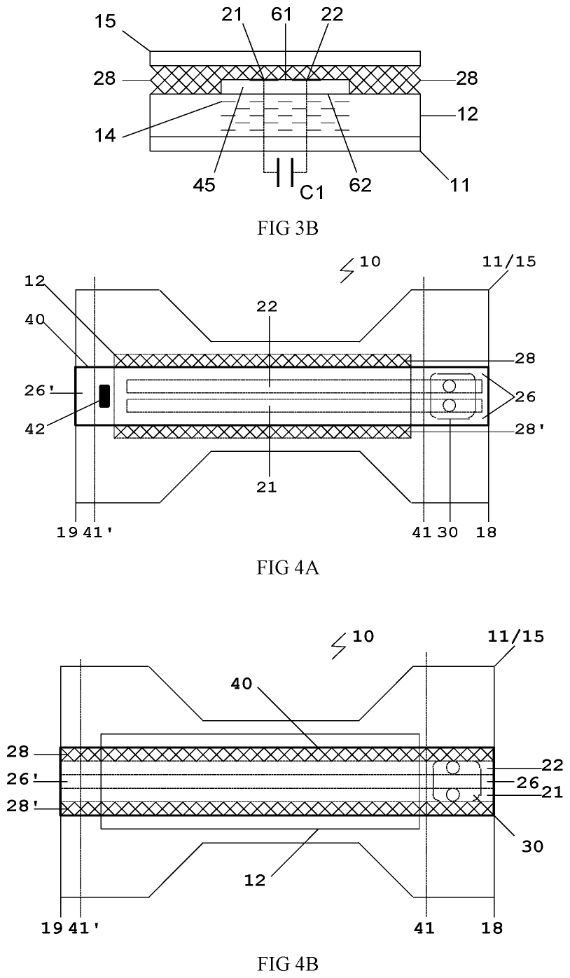

FIG. 3A and FIG. 3B are the A-A′ cross section structure diagram and B-B′ cross section structure diagram/equivalent circuit diagram related to FIG. 1 of a smart absorbent article adopting a single-sided sensing film as an embodiment of the present disclosure. In this embodiment, the first and second sensing lines 21 and 22 are arranged on the plastic film substrate 45 to form a single-sided sensing film and are arranged between the diaper topsheet 11 and the backsheet 15. In the A-A′ section (FIG. 3A), it can be seen that the contact surface 61 faces towards the backsheet 15 and includes a separable part between it and the backsheet to form a built-in pocket 26. The sensor 30 can be arranged in the built-in pocket 26, the side provided with contact points 31 and 32 is closely attached to the contact surface 61 and is in face to face with the exposed sensing lines 21 and 22 on the contact surface and is electrically connected.

In this embodiment, the built-in pocket 26 is near the diaper front edge 18, where no absorbent layer 12 exists, so the topsheet 11 is directly attached to the sensing surface 62, and the part beyond the sensing surface/sensing film in width is bonded to the backsheet 15 by adhesive 28 (including hot melt adhesive, structural adhesive, etc.) and forms the bonding boundary of the built-in pocket 26. It not only prevents moisture from penetrating into the contact surface 61 and causing short circuit of sensing lines 21 and 22, but also effectively fixed the sensing film, and automatically kept the width of the built-in pocket 26 consistent with the width of the plastic film substrate 45, and automatically positioned and aligned between the sensing film and the built-in pocket. It ensures that the contacts 31 and 32 of the sensor inserted into the pocket can be aligned with the sensing lines 21 and 22 on the contact surface 61 and achieve a reliable electrical connection.

In this embodiment, the built-in pocket 26 is arranged under the front belly patch 25, and at least part of the built-in pocket 26 is within the orthographic projection range of the front belly patch 25. When the diaper is in the wearing state, the elastic force generated on the waist can be translated into the force of the built-in pocket 26 against the front abdomen of the user (downward force shown in the figure) by applying left and right stickers 16 and 17, which also compacts the sensor 30 in the pocket and makes the contact with the sensing lines more reliable. When the diaper is not in the wearing state, the force attached to the user's front abdomen will automatically disappear and make the sensor 30 easy to take out, which is a technical feature of the embodiment of the present disclosure by setting the built-in pocket under the front belly patch, which cannot be done by setting the pocket on the outer surface of the diaper.

In the B-B′ section (FIG. 3B), it can be seen that the absorbent layer 12 and the sensing surface 62 are attached together, and the sensing lines 21 and 22 can detect the moisture on the absorbent layer 12 through the plastic film substrate 45 as the dielectric layer and by the means of electrolytic capacitance, thus realizing the wetness detection function of diapers. The degree of wetness is proportional to the electrolytic capacitance C1. Since there is no built-in pocket 26 on this section, the contact surface 61 is bonded to the backsheet 15 by adhesive 28, which effectively prevents the moisture in the absorbent layer 12 from penetrating into the contact surface 61 and short-circuiting the sensing lines 21 and 22. In this embodiment, the backsheet 15 is covered on the contact surface 61 of the entire sensing film, so the backsheet 15 can be regarded as a waterproof covering layer and constitutes an integral part of the short-circuit protection mechanism of the embodiment of the present disclosure.

FIG. 4A and FIG. 4B are the schematic diagram of the appearance structure of a smart absorbent article using the single-sided sensing film when it is used with a sensor in an embodiment of the present disclosure, which includes a diaper, a single-sided sensing film 40, a built-in pocket 26, a short-circuit protection mechanism, etc. The diaper consists of a topsheet 11, an absorbent layer 12 and a backsheet 15. The topsheet 11 and the backsheet 15 are overlapped as they are the same size in this embodiment. The contact surface 61 of the sensing film 40 is closely attached to the backsheet 15, and the sensing surface 62 of the sensing film 40 faces the absorbent layer. The sensing lines 21 and 22 can detect the wetness state of the absorbent layer 12 by means of electrolytic capacitance.

In this embodiment, the backsheet 15 is used as a waterproof covering layer to implement short circuit protection on the sensing lines 21 and 22 on the contact surface 61. In addition, the front and back edges 18 and 19 of the diaper between the contact surface 61 and the backsheet 15 also include a mutually separable part to form a built-in pocket 26 and 26′. Among them, a functional pocket 26 is formed at the front edge 18 of the diaper, which is used to insert a sensor and make the sensor contacts electrically connected with the sensing lines, while a redundant pocket 26′ is formed at the back edge 19, which can provide error redundancy for the positioning cut during the smart diaper production. In the production process of diapers, each pair of diapers was originally connected end to end, and it was not until the later stage of production that the two adjacent diapers were cut off and an independent finished diaper was obtained. The original built-in pocket was divided into two parts, which were respectively located at the front and back edges of the diaper and formed pockets 26 and 26′.

In the embodiment shown in FIG. 4A, there is no sensing lines within the redundant pocket 26′, so that the normal operation of sensing lines 21 and 22 will not be affected even if there is a liquid wetting the redundant pocket 26′, which can also be considered as part of the short-circuit protection mechanism. Since the sensing lines are asymmetrical, in order to place the sensing film in the correct position of the diaper, the sensing film 40 also includes a color positioning marker 42. As for the embodiment shown in FIG. 4B, the sensing film is completely symmetrical, in this case there is no need for positioning, as long as the sensing film is placed in the diaper on a specific layer for use. The symmetrical structure can increase the versatility and convenience of the sensing film, which can adapt to the production of smart diapers of different specifications and lengths.

In the production process of smart diapers, the sensing film 40 is usually pasted on the backsheet 15, then the absorbent layer 12 is covered on the sensing film 40 and bonded, and then the topsheet 11 is covered on the absorbent layer 12 and bonded, and the topsheet beyond the absorbent layer 12 will be directly bonded with the backsheet 15. All of this bonding creates a bonding boundary that prevents moisture from seeping in. For example, in FIG. 4A, the bonding zone 28/28′ is generated between the absorbent layer 12 and the backsheet 15, which forms a bonding boundary on each side of the sensing film 40. Another example is the bonding zone 28/28′ in FIG. 4B, which is generated by the adhesion between the edges of both sides of the sensing film and the backsheet 15. In addition to forming the adhesive boundary on both sides of the sensing film, an adhesive free and separable hollow part is also generated between the contact surface 61 and the backsheet 15, which constitutes a built-in pocket that runs from end to end. It creates a functional pocket 26 and a redundant pocket 26′ at the front and back ends of the diaper.

In practical applications, the sensing film can also be bonded to the backsheet 15 in areas outside the range of the built-in pocket 26/26′ (e.g., from 41 to 41′). These adhesives allow for more effective protection of the sensing lines 21 and 22. In the present embodiment, the area inside the built-in pocket, if sensing lines are present, can constitute a contact area for contacting and implementing electrical connection with the contacts of the inserted sensor; In the area outside the built-in pocket, if there is sensing lines (such as the area from 41 to 41′ in FIG. 4B), it can constitute a sensing area, which is mainly used for wetness detection of the diaper.

FIG. 5A and FIG. 5B are three-dimensional structure diagram and cross section structure diagram/equivalent circuit diagram of a composite sensing film suitable for the smart absorbent article of an embodiment of the present disclosure. Different from the single-sided sensing film mentioned above, at least part of the sensing lines in this embodiment is located in a waterproof sandwich composed of two waterproof materials and constitutes a composite film, so it can be called a composite capacitive sensing film. For example, in this embodiment, in addition to the plastic film substrate 45 and the first and second sensing lines 21 and 22, a waterproof protective layer 46 covers the sensing lines 21 and 22 and together with the plastic film substrate 45 forms a waterproof sandwich in which the sensing lines are sandwiching. The waterproof protective layer 46 of the embodiment of the present disclosure includes a polymer waterproof coating or a polymer waterproof composite layer/covering layer.

The area 47 covered by the plastic film substrate 45 and the waterproof protective layer 46 is the sensing area of the sensing film. The sensing lines can detect wetness on outer surface 62 of the plastic film substrate 45 and on outer surface 63 of the waterproof protective layer 46. Therefore, the composite sensing film in this embodiment has the capability of double-sided detection. In practical applications, the side facing the liquid to be detected is generally regarded as the sensing surface, and the insulating material separating the sensing lines and the sensing surface is regarded as the dielectric layer.

When liquid 14A containing electrolyte is accumulated on the sensing surface 62, electrolytic capacitor C1 is generated between the sensing lines 21 and 22, and its capacitance is inversely proportional to the thickness of the plastic film substrate 45, proportional to the dielectric constant of the plastic film substrate 45, and proportional to the area of the liquid 14A corresponding to the electrodes (sensing lines). When liquid 14B containing electrolyte is accumulated on the sensing surface 63, electrolytic capacitor C2 will be generated between the sensing lines 21 and 22, and its capacitance is inversely proportional to the thickness of the waterproof protective layer 46, proportional to the dielectric constant of the waterproof protective layer 46, and proportional to the area of the liquid 14B and the electrodes (sensing lines).

In the present embodiment, the area 43 of the sensing lines without waterproof protective layer 46 is the contact area of the sensing film, and the sensing lines 21 and 22 are exposed at the contact area 43 and constitute a contact surface 61, which can be used for face-to-face contact with the contacts of the sensor and electrical connected. In this embodiment, the orientation of the contact surface 61 and the sensing surface 63 is consistent, and since the waterproof protective layer 46 is very thin, the contact surface 61 and the sensing surface 63 can be considered to be on the same plane, but different in location/area.

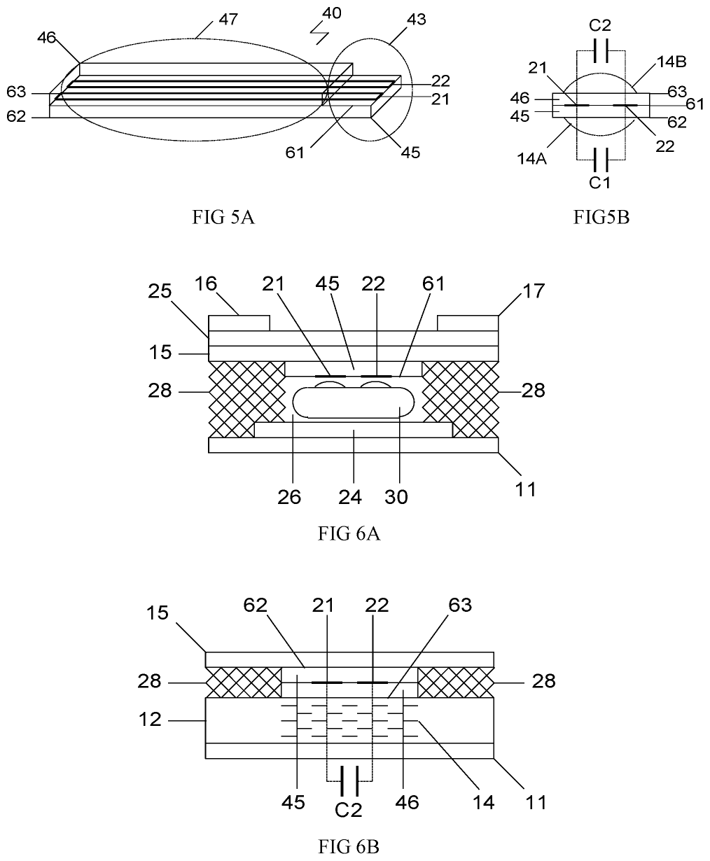

FIG. 6A and FIG. 6B are the A-A′ cross section structure diagram and B-B′ cross section structure diagram/equivalent circuit diagram related to FIG. 1 of the smart absorbent article adopting a composite sensing film as an embodiment of the present disclosure. The sensing film in the figure includes a plastic film substrate 45, a contact surface 61, a sensing surface 62, a waterproof protective layer 46, a sensing surface 63, a first sensing line 21, a second sensing line 22 and other components.

In the A-A′ cross section (FIG. 6A), the embodiment also includes a waterproof covering layer 24 covering the contact area of the sensing film and corresponding to the contact surface 61 of the sensing film and includes mutually separable parts between the contact surface 61 and the waterproof covering layer 24 and forming a built-in pocket 26. In order to prevent the moisture in the absorbent layer 12 from entering the built-in pocket 26 and short-circuit the sensing lines 21 and 22, the waterproof covering layer 24 needs to have good waterproof performance, and can usually be composed of plastic film, waterproof paper, water-repellent non-woven fabrics, or other materials. The width of the waterproof covering layer 24 is usually not less than that of the plastic film substrate 45 so that the entire contact surface 61 can be effectively covered.

The topsheet 11 is overlaid on the waterproof covering layer 24, the part of which beyond the waterproof covering layer 24 is bonded to the backsheet 15 by adhesive 28, and the part of the waterproof covering layer 24 beyond the plastic film substrate 45 in width is also bonded to the backsheet 15, All this bonding not only creates a boundary that prevents moisture from penetrating, but also automatically aligns the width of the built-in pocket 26 with the width of the sensing film/plastic film substrate 45, which is a technical feature and beneficial effect of the embodiment of the present disclosure using the sensing film to create a built-in pocket.

In this embodiment, the outer surface 62 of the plastic film substrate 45 is oriented towards the backsheet 15. Since there is no liquid between the plastic film substrate 45 and the backsheet 15, the plastic film substrate 45 mainly plays the role of carrying and protecting the sensing lines in this embodiment. In the B-B′ section (FIG. 6B), the absorbent layer 12 is seen over the waterproof protective layer 46, and the part beyond the waterproof protective layer 46 in width will be bonded to the backsheet 15. The outer surface 63 of the waterproof protective layer 46 faces towards the absorbent layer and forms the sensing surface. Therefore, the waterproof protective layer 46 acts as a dielectric layer separating and insulating the sensing lines from the sensing surface in this embodiment. The sensing lines detects the moisture of the absorbent layer 12 through the waterproof protective layer 46 by means of electrolytic capacitance and realizes the wetness detection function against the absorbent layer of the diaper.

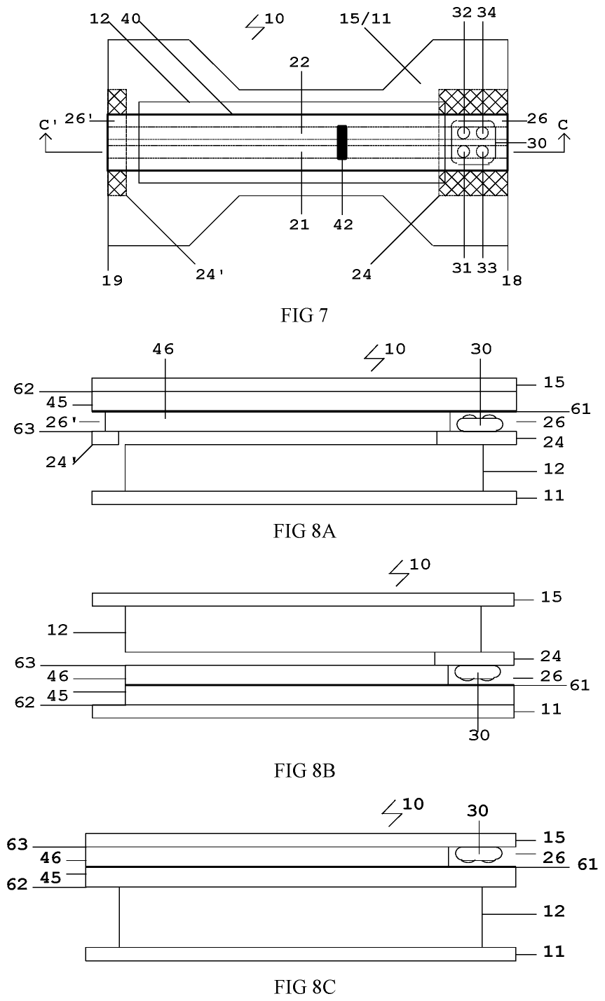

FIG. 7 is a schematic diagram of the appearance structure of a smart absorbent article using a composite sensing film when it is used together with a sensor in an embodiment of the present disclosure, which includes a diaper, a composite sensing film 40, a built-in pocket 26, a short-circuit protection mechanism and other components, wherein the diaper includes a topsheet 11, an absorbent layer 12 and a backsheet 15. The sensing film 40 includes the first and the second sensing lines 21 and 22. The part of the sensing film that is not covered by the waterproof covering layer 24/24′ constitutes the sensing zone. Sensing lines 21 and 22 can detect the moisture state of the diaper through the sensing surface of the sensing film 40.

The waterproof covering layer 24/24′ is covered on the contact surface of the sensing film, which includes a separable part between the waterproof covering layer and the contact surface and constitutes a functional pocket 26 and a redundant pocket 26′ respectively, wherein the functional pocket 26 is located at the front end of the diaper, the opening is flush with the front edge 18, and the sensor 30 can be inserted through the opening. Sensor contacts 31 and 33 are electrically connected to sensing line 21, while sensor contacts 32 and 34 are electrically connected to sensing line 22. As for the redundant pocket 26′ is located at the rear edge 19 of the diaper, which mainly provides redundancy for the cutting error of the diaper, while the positioning mark 42 can provide positioning for the production of smart diapers. As for the part of the waterproof cover 24/24′ beyond the contact surface, it will bond with the backsheet 15 and form a boundary that prevents the absorbent layer moisture from penetrating into it, as shown in the shaded part in the figure.

In the present embodiment, contacts 31, 33 (or contacts 32, 34) may constitute an insertion detection device, which can be triggered when the sensor 30 is inserted into the built-in pocket 26 and the two contacts contact the same sensing line at the same time and cause the sensor to enter the working state; When the sensor 30 is taken out of the pocket 26, the insertion detection device can be masked, so that the sensor enters the sleep/shutdown state to save power.

FIG. 8A, FIG. 8B, and FIG. 8C are the C-C′ cross section structure diagrams related to FIG. 7 of the smart absorbent article using the composite sensing film for embodiments of the present disclosure, which are some concrete examples of the application of the composite sensing film to smart diapers. The figure includes a diaper composed of the topsheet 11, the absorbent layer 12, and the backsheet 15, as well as the composite sensing film 40 arranged inside the diaper. The sensing film includes the plastic film substrate 45 and the waterproof protective layer 46, which constitute the waterproof sandwich to protect the sensing lines 21 and 22. The sensing lines can detect the wetness state of the diaper through the plastic film substrate 45 or the waterproof protective layer 46 by means of electrolytic capacitance.

Since no waterproof protective layer 46 exists on the contact surface 61 of the sensing film, the sensing lines are exposed on the contact surface. In order to protect the sensing lines, the waterproof covering layer 24 can be covered on the contact surface 61, and the two include mutually separable parts and constitute the built-in pocket 26 (as shown in FIG. 8A and FIG. 8B). In this case, the waterproof covering layer 24 plays a role in preventing the moisture in the absorbing layer 12 from penetrating into the contact surface 61 and constitutes the short-circuit protection mechanism of the embodiment of the present disclosure.

In practical application, the sensing film 40 can be arranged between the backsheet 15 and the absorbent layer 12 (as shown in FIG. 8A and FIG. 8C), and between the topsheet 11 and the absorbent layer 12 (as shown in FIG. 8B). At the same time, the outer surface 63 of the waterproof protective layer 46 can be oriented towards the absorbent layer 12 to form the sensing surface (as shown in FIG. 8A and FIG. 8B), and the outer surface 62 of the plastic film substrate 45 can be oriented towards the absorbent layer to form the sensing surface (as shown in FIG. 8C). In addition to using the waterproof covering layer 24 to cover contact surface 61 to form the built-in pocket 26 (as shown in FIG. 8A and FIG. 8B), the backsheet 15 of the diaper can also be used as the waterproof covering layer to cover contact surface 61, and a separable part between the backsheet 15 and contact surface 61 is included to form the built-in pocket 26 (as shown in FIG. 8C).

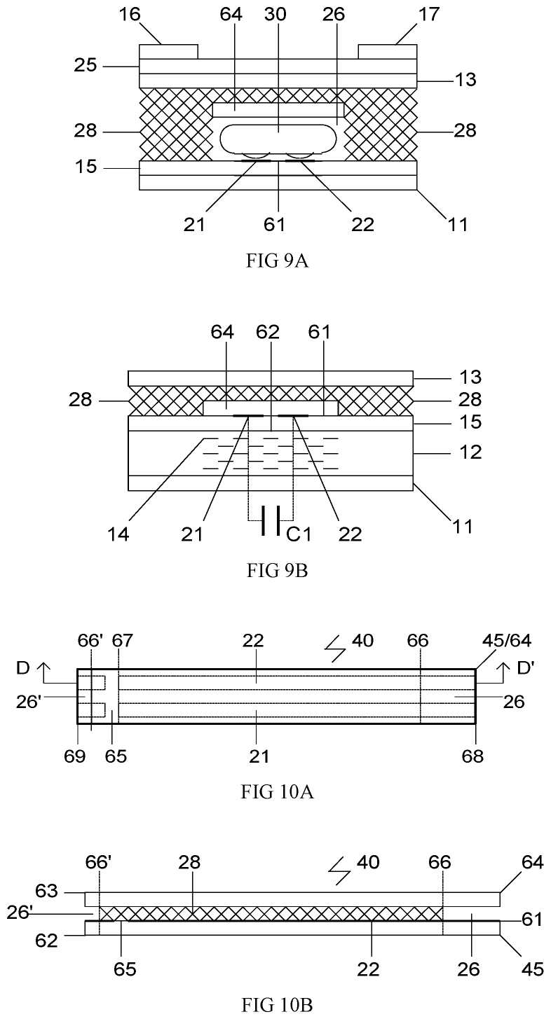

FIG. 9A and FIG. 9B are the A-A′ cross section structure diagram and B-B′ cross section structure diagram/equivalent circuit diagram related to FIG. 1 of an embodiment of the present disclosure of a smart absorbent article using a composite backsheet type sensing film. Unlike the other aforementioned embodiments where the sensing lines are arranged between the backsheet and the topsheet of the diaper, the sensing lines 21 and 22 in this embodiment are arranged on the outer surface of the backsheet 15 (backward absorbent layer). In order to protect the sensing lines, an outer non-woven fabric 13 is covered on the outer surface of the back sheet 15 in this embodiment, so that the outer non-woven fabric 13 plays the role of a waterproof covering layer, and the outer non-woven fabric 13 and the backsheet 15 are combined together through an adhesive to form a composite backsheet, so the sensing film of the embodiment can be called a composite backsheet type sensing film.

In order to accurately control the size of the built-in pocket 26, it can be seen in the A-A′ section (FIG. 9A) that an additional waterproof covering layer 64 is included between the backsheet 15 and the outer non-woven fabric 13. In the actual production process of smart diapers, the sensing lines 21 and 22 are generally printed on the outer surface of the backsheet 15 to form a contact surface 61, and then the waterproof covering layer 64 is affixed to the sensing lines 21 and 22. The waterproof covering layer 64 and the contact surface 61 includes a separable part between the waterproof covering layer 64 and the contact surface 61 and constitute a built-in pocket 26. The outer non-woven fabric 13 is then covered on the waterproof covering layer 64 and bonded to each other by adhesive 28, and the part beyond the waterproof covering layer 64 in width is bonded to the backsheet 15, which in addition to forming a boundary that prevents liquid penetration and constitutes a short-circuit protection mechanism, also makes the width of the built-in pocket 26 automatically consistent with the width of the waterproof covering layer 64.

The waterproof covering layer 64 of this embodiment is optional. If the waterproof covering layer 64 is not used, it is necessary to create a non-sprayed area between the outer non-woven fabric 13 and the backsheet 15 during the diaper production process to form a separable part and thus constitute a built-in pocket 26. In this case, the outer non-woven fabric 13 can be regarded as a waterproof covering layer and acts as a short-circuit protection mechanism. It should be noted that the local process spray usually has a certain divergence, and the size of the built-in pocket generated by it may have a certain error, but it can be used in the case of low requirements.

In this embodiment, the other side of the backsheet 15 without the presence of sensing lines (inner surface 62) would face the absorbent layer and act as the sensing surface, while the backsheet 15 itself would act as the dielectric layer insulating the sensing lines 21 and 22 from the sensing surface 62. When there is a liquid 14 containing electrolyte in the absorbent layer 12, the liquid 14 will form the electrolytic capacitor C1 together with the dielectric layer 15, the sensing lines (electrode) 21 and 22. As long as the value of C1 is detected, the wetness state on the sensing surface can be known, thus realizing the wetness state detection function of the diaper.

The outer non-woven fabric 13 of this embodiment is an integrated cut intact non-woven fabric, including composite non-woven fabric, hydrophobic non-woven fabric, water-repellent non-woven fabric, and other materials. The built-in pocket 26 can be arranged at the front end of the diaper and extended to the edge, thereby creating an invisible opening at the front edge. The sensor 30 can be inserted into the built-in pocket 26 through the invisible opening. In this embodiment, the influence of the color of the sensing lines on the appearance of the diaper can be reduced or eliminated because the sensing lines 21 and 22 are covered with the outer non-woven fabric 13 and the waterproof covering layer 64, and the influence of the external moisture of the diaper on the sensing lines can be eliminated to avoid the occurrence of false alarms.

FIG. 10A and FIG. 10B are the front structure diagram and D-D′ cross section structure diagram of a capacitive sensing film including a head-and-tail built-in pocket suitable for used in a smart absorbent article as an embodiment of the present disclosure. In the aforementioned embodiments, the sensing film itself has no pockets and needs to be combined with other materials, such as the waterproof covering layer 24, the backsheet 15 or the outer non-woven fabric 13, to form the built-in pocket. In present embodiment, a waterproof covering layer is integrated into the sensing film in advance to form a sensing film with its own pocket and short-circuit protection mechanism, which can facilitate the application of the sensing film and the production of the smart diapers.

The sensing film 40 of the present embodiment includes a plastic film substrate 45, sensing lines 21/22 arranged on one side of the plastic film substrate, and a waterproof covering layer 64 covering the sensing lines, which fully protects the sensing lines 21 and 22. The waterproof covering layer 64 and the plastic film substrate 45 include both bonded part (at middle section) and separable part (at ends of the head and tail), wherein a separable part included at the front edge 68 constitutes a functional pocket 26, and a separable part included at the rear end edge 69 of the sensing film constitutes a redundant pocket 26′. Sensing lines 21 and 22 are exposed in the two pockets, and the plane where the sensing lines are located constitutes a contact surface 61, so as to contact and implement electrical connection with the contacts of the sensor inserted in the pocket, and the corresponding area constitutes a contact area of the sensing film.

From the bottom boundary 66 of pocket 26 to the bottom boundary 66′ of pocket 26′, the waterproof covering layer 64 is bonded together with the plastic film substrate 45, which forms part of the short-circuit protection mechanism and forms a sensing area of the sensing film, which can be used to detect wetness on the outer surface 62 of the plastic film substrate 45 or the outer surface 63 of the waterproof covering layer 64. A breakpoint 65 is also included in this embodiment to make the head and tail ends of the sensing lines 21 and 22 disconnected from each other to prevent the normal operation of the sensing lines in the functional pocket 26 from being affected by short circuits in the sensing lines in the redundant pocket 26′. In addition, breakpoint 65 can also be used as a positioning marker to help set the sensing film in the appropriate position of the diaper.

When the sensing film is set inside the diaper for use, the front edge 68 will usually be flush with the front edge 18 of the diaper, and the sensing area may correspond to the absorbent layer of the diaper. The embodiment may either use the plastic film substrate 45 as the dielectric layer and the outer surface 62 as the sensing surface or use the waterproof covering layer 64 as a dielectric layer and the outer surface 63 as the sensing surface. Sensing lines 21 and 22 can detect the wetness state of the sensing surface through the corresponding dielectric layer by means of electrolytic capacitance, and the detection sensitivity (the ability to generate electrolytic capacitance) is proportional to the dielectric constant of the dielectric layer and inversely proportional to the thickness of the dielectric layer.

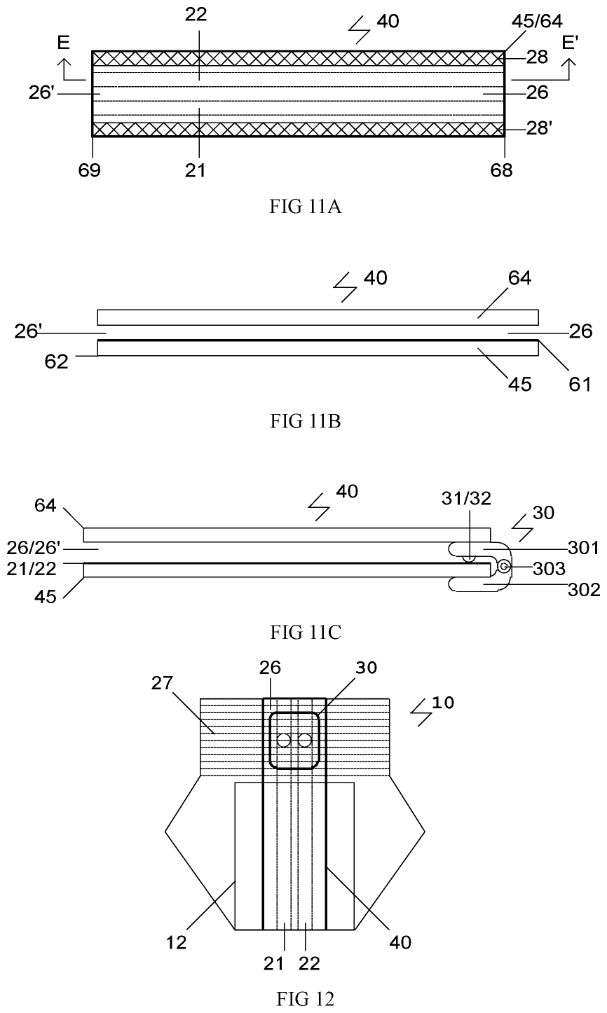

FIG. 11A, FIG. 11B and FIG. 11C are the front structure diagram, the E-E′ cross section structure diagram and the position relationship diagram of a capacitive sensing film of an embodiment of the present disclosure, this kind of sensing film includes a through-type built-in pocket and is suitable for use in the smart absorbent article of the embodiment of the preset disclosure. Unlike the embodiments shown in FIG. 10A and FIG. 10B, which are bonded in the middle section of the sensing film, the plastic film substrate 45 and the waterproof covering layer 64 in this embodiment are bonded at the edges of both sides, forming bonding zones 28 and 28′ respectively, which can be bonded by adhesive or hot pressing. When hot pressing bonding is selected, EVA, TPU, PES, PVC or other hot melt adhesive film can be selected as waterproof covering layer. In this embodiment, the edges of the two sides of the sensing film are bonded together to form a boundary that prevents liquid penetration and constitutes a short-circuit protection mechanism of the embodiment of the present disclosure, so that the sensing lines 21 and 22 are effectively protected, and the hollow part constitutes a through-type built-in pocket, and the through-type pocket includes a pocket with opening at the left and right ends of the sensing film respectively, which are a functional pocket 26 and a redundant pocket 26′.

In this embodiment, the side of the plastic film substrate 45 with sensing lines 21 and 22 is the contact surface 61, and the other side of the plastic film substrate 45 where the sensing lines does not exist is the sensing surface 62, the plastic film substrate 45 constitutes the dielectric layer, and the sensing lines 21 and 22 detect the wetness state on the sensing surface 62 through the dielectric layer 45 by means of electrolytic capacitance. The sensing film in this embodiment is not divided into a sensing area and a contact area, or the sensing film in any segment contains both the sensing area and the contact area. The sensing film in this embodiment is itself equipped with the built-in pockets and the short-circuit protection mechanism and can be easily set for use on any layer of the diaper. At the same time, because the sensing film in this embodiment is completely symmetrical, it does not need to be positioned in the production process of smart absorbent articles, and these characteristics make it more versatile and convenient in application.

The sensing film of this embodiment can be cut off at any length for use, e.g. it may be selected to be as long as the diaper so that the front edge 68 is flush with the front edge 18 of the diaper, or it may be selected to be longer than the diaper so that the front edge 68 protrudes from the front edge 18 of the diaper so that the sensor can be inserted for use from the protruding part. In order to facilitate production, the sensing film can also be packed into a large roll of coil form, each roll of sensing film can produce thousands of smart diapers, making the sensing film to be a general smart diaper production raw material.

Since the pocket of this embodiment is a through-type pocket, in order to enable the sensor to be fixed in the pocket, a clamshell-type design as shown in FIG. 11C can be used. The sensor 30 in the figure includes an insert section 301 and a clamshell section 302, both of which are hinged by hinge 303. When using, you can first open the clamshell 302, and then insert the insert section 301 into the built-in pocket 26, so that contact 31/32 and sensing lines 21/22 face to face contact, and then close the clamshell 302 to clamp the plastic film substrate 45, which not only makes the contact between the sensor contact and the sensing lines more reliable, also prevents the sensor from slipping out or into deeper positions. The sensor design is applicable not only to the present embodiment, but also to other embodiments of the present disclosure.

FIG. 12 is a schematic diagram of the appearance structure of the smart absorbent article including a built-in pocket on the elastic waist of an embodiment of the present disclosure. The difference from the diapers in the other embodiments above is that the diaper in the present embodiment is a panty diaper with an elastic waistline 27, such as pull-ups, training pants, etc. The built-in pocket 26 is arranged in the interlayer of the elastic waistline 27. When the diaper is in the wearing state, the elastic force generated on the elastic waistline can be converted into the vertical force pressing the built-in pocket 26 to the user's body, which can effectively press and fix the sensor 30 in the pocket 26. The contact point of the sensor 30 and the sensing lines 21 and 22 on the contact surface of the sensing film 40 are made more reliable.

The sensing area of the sensing film 40 in this embodiment corresponds to the absorbent layer 12 of the diaper, while the contact area of the sensing film 40 is located within the built-in pocket 26 of the elastic waist of the diaper, that is, the two are separated from each other in position to further prevent the moisture in the absorbent layer 12 from penetrating into the contact area and causing short circuit of the sensing lines, which can also be regarded as a short-circuit protection mechanism. The sensing film 40 may adopt any of the above embodiments, and the built-in pocket 26 may extend to the edge of the diaper waist and include in the sandwich gap at the edge an opening to the built-in pocket through which a sensor 30 performing wetness detection can be inserted.

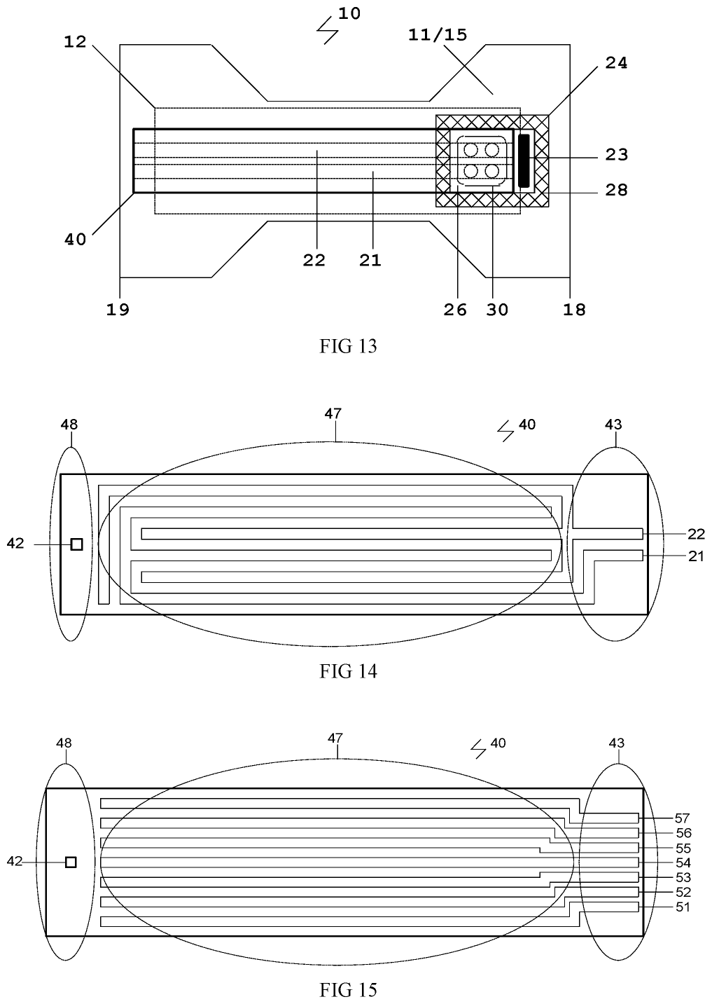

FIG. 13 is a schematic diagram of the appearance structure of a smart absorbent article as an embodiment of the present disclosure, whose outer face includes an incision leading to a built-in pocket. In the aforementioned embodiments, the opening of the functional pocket 26 is generally located at the front edge of the diaper, and the sandwich gap between the contact surface 61 of the sensing film and the backsheet 15 or the waterproof covering layer 24/64 is used as an invisible opening. In practical applications, the sensor can also be placed into the built-in pocket by cutting a notch on the outer or inner surface of the diaper.

The smart diaper 10 in the present embodiment includes a topsheet 11, an absorbent layer 12, a backsheet 15, a sensing film 40, a waterproof covering layer 24 and other components. The sensing film includes sensing lines 21 and 22, and the contact surface of the sensing film 40 includes a mutually separable part between the waterproof covering layer or the backsheet and constitutes a built-in pocket 26, an incision 23 is also included on the outer surface of the diaper or on the inner surface at the front edge, and the incision 23 leads to the built-in pocket 26 through which the sensor 30 can be placed into the built-in pocket 26 for use.

FIG. 14 and FIG. 15 are schematic diagrams of the appearance structure of a sensing film having a toroidal sensing lines design and a plurality of sensing lines designed for the smart absorbent article of the embodiments of the present disclosure. The sensing film 40 in the figure includes a contact area 43, a sensing area 47 and a redundant area 48. In application, the contact area 43 corresponds to the front edge of the diaper and includes bare sensing lines and constitutes a contact surface where a functional pocket can be generated for contact and electrical connection with the inserted sensor contacts; Redundancy area 48 corresponds to the back edge of the diaper, where redundant pockets are generated and redundancy is provided for the diaper cutting; The sensing area 47 corresponds to the position of the crotch of the diaper and is used to detect the wetness state of the diaper

In the embodiment shown in FIG. 14, the sensing area 47 includes a curved sensing lines design, which includes 6 sensing lines from top to bottom, in which singular sensing lines are connected with the sensing line 22, and even number of sensing lines are connected with the sensing line 21, this method can effectively expand the coverage area and detection range of sensing area 47. The sensing area 47 of the embodiment shown in FIG. 15 consists of seven parallel sensing lines (from 51 to 57), which is also designed to effectively extend the coverage of the sensing lines and provide more wetness status information at the location. It should be noted that these sensing line patterns in this embodiment are asymmetrical from end to end. In order to enable the sensing film to be accurately embedded in the specific position of the diaper, a positioning mark 42 is also included on the sensing film 40.

In addition, the sensing film of the embodiment of the present disclosure can also have more structural forms and more sensing line pattern designs, and corresponding changes can be made on the basis of the above-mentioned embodiments combined with actual needs. In addition, in the aforementioned embodiments, the built-in pocket is arranged on the front straight face of the diaper, and in practical application, it can also be arranged on other positions of the diaper, such as the crotch, back or side position of the diaper, as long as there is a sensing film/sensing lines through these places.

Because the smart absorbent article 10 of the embodiments of the present disclosure has a smart wetness detection function, a smart wetness state monitoring system can be constituted by inserting a sensor 30 into the built-in pocket 26. In such a monitoring system, the sensor will include at least two contacts for contact and electrical connection with the first and second sensing lines, and the sensor will also include a capacitance detection device to detect the wetness state of the absorbent article by means of electrolytic capacitance. In practical applications, the sensor preferably has four or more contacts, two of which can form an insertion detection device. The sensor can also usually include a wireless transmitter, and a wireless receiving device can be set up to accompany it, so that the wetness status information of the absorbent article can be sent and received wirelessly, and the wireless receiving device usually includes an audible and visual alarm device, a smart phone, or a personal computer.

The foregoing descriptions are merely exemplary embodiments of the present disclosure, and certainly are not intended to limit the scope of claims of the present disclosure. Therefore, equivalent variations made in accordance with the claims of the present disclosure shall fall within the scope of the present disclosure.

1. A smart absorbent article based on a capacitive sensing film and a built-in pocket, comprising a disposable absorbent article, a capacitive sensing film, a built-in pocket and a short-circuit protection mechanism, the disposable absorbent article comprises a topsheet, an absorbent layer and a backsheet, and the sensing film comprises a contact surface, a sensing surface, a dielectric layer and at least two sensing lines, the outer surface of the dielectric layer constitutes the sensing surface, the dielectric layer separates and insulates the sensing lines and the sensing surface, when the sensing surface is soaked by a liquid containing electrolyte, the sensing lines can realize the wetness detection function of the sensing surface through the dielectric layer by means of electrolytic capacitance, the built-in pocket is adapted to insert a sensor, the contact surface facilitates face-to-face contact and electrical connection between the sensor and the sensing lines, the short-circuit protection mechanism is adapted to protect the sensing lines from moisture inside and outside the disposable absorbent article,
wherein at least part of the sensing lines is located within the contact surface and includes exposed part, the short-circuit protection mechanism comprises a waterproof covering layer which covers the contact surface, and a separable part between the contact surface and the waterproof covering layer constitutes the built-in pocket, the sensing lines can realize the wetness detection function of the absorbent article through the dielectric layer by means of electrolytic capacitance; and
the capacitive sensing film comprises a sensing film with a head-and-tail built-in pocket, the sensing film comprises a plastic film substrate, the sensing lines are arranged on one surface of the plastic film substrate, the waterproof covering layer is covered on the sensing lines, the waterproof covering layer and the plastic film substrate include a mutually bonded part in the middle section and constitute a sensing area, and include a mutually separable part at both ends of the head and tail and constitute the built-in pocket, the outer surface of the plastic film substrate or the waterproof covering layer is oriented towards the absorbent layer; or
the capacitive sensing film comprises a sensing film with a through-type built-in pocket, the sensing film comprises a plastic film substrate, the sensing lines are arranged on one surface of the plastic film substrate and constitutes the contact surface, the other surface of the plastic film substrate constitutes the sensing surface, the plastic film substrate constitutes the dielectric layer, the waterproof covering layer is covered on the contact surface, the edge of both sides of the waterproof covering layer and the plastic film substrate includes a part bonded to each other and constitutes a boundary preventing liquid penetration into, a hollow port is further included between the waterproof covering layer and the plastic film substrate and constitutes the through-type built-in pocket.

2-3. (canceled)

4. The smart absorbent article according to claim 1, wherein the sandwich at the front edge of the disposable absorbent article comprises an invisible opening, which leads to the built-in pocket for inserting a sensor to achieve the wetness detection function.

5. (canceled)

6. The smart absorbent article according to claim 1, wherein the disposable absorbent article comprises an elastic waist circumference, the built-in pocket is arranged on the elastic waist circumference, when the smart absorbent article is in a wearing state, the elasticity generated on the elastic waist circumference can be converted into the force of the built-in pocket attached to the user's body, the sensor arranged in the built-in pocket is thus compressed and the contact of the sensor with the sensing lines is made more reliable.

7. The smart absorbent article according to claim 1, wherein the disposable absorbent article comprises a diaper, an insertion pad, a sanitary napkin, a maternal towel or an urine pad, the topsheet comprises a hydrophilic non-woven fabric, the absorbent layer comprises polymer absorbent material, the backsheet comprises a breathable or impermeable PE film, the sensing lines comprise ink lines printed with conductive ink, metal lines formed by physical vapor deposition, aluminum foil or copper foil, the plastic film substrate comprise a PE, PP, CPP, BOPP or PET film, and the waterproof covering layer comprises a plastic film, a waterproof paper or a water-repellent non-woven fabric, and the electrolyte containing liquid comprises urine, loose stool, sweat or blood containing salt.

8. (canceled)

9. A capacitive sensing film including a head-and-tail built-in pocket for making the smart absorbent article described in claim 1, comprising a plastic film substrate, a waterproof covering layer and at least two sensing lines, the sensing lines are arranged on one side of the plastic film substrate, the waterproof covering layer covers the sensing lines, the waterproof covering layer and the plastic film substrate include a mutually bonded part in the middle part to form a protected sensing area for preventing liquid penetration into the area and causing a short circuit of the sensing lines, the waterproof covering layer and the plastic film substrate include a mutually separable part at both ends of the head and tail and constitute a contact area, wherein the contact area located at the front edge constitutes a functional pocket, the sensing lines are exposed in the functional pocket and constitutes a contact surface for face-to-face contact and electrical connection with the contact points of the sensor inserted into the pocket, and the contact area located at the back end edge constitutes a redundant pocket, which can provide error redundancy for the positioning cutting during the production of the smart absorbent article; and
the plastic film substrate or the waterproof covering layer constitutes the dielectric layer, the outer surface of which constitutes the sensing surface, the sensing lines can detect the wetness state of the sensing surface through the dielectric layer and by means of electrolytic capacitance, and the detection sensitivity is proportional to the dielectric constant of the dielectric layer and inversely proportional to the thickness of the dielectric layer.

10. (canceled)

11. A capacitive sensing film including a through-type built-in pocket for making the smart absorbent article described in claim 1, comprising a plastic film substrate, a waterproof covering layer and at least two sensing lines, the sensing lines are arranged on one side of the plastic film substrate, the waterproof covering layer covers the sensing lines, both sides of the waterproof covering layer and the plastic film substrate include a part at the edge that is bonded to each other and thus generates a boundary that prevents liquid penetration and constitutes a short-circuit protection mechanism, the waterproof covering layer and the plastic film substrate comprises a hollow part and constitutes a through-type built-in pocket, the sensing lines are exposed in the built-in pocket and constitutes the contact surface for face-to-face contact and electrical connection with the contacts of a sensor inserted into the built-in pocket; and
the plastic film substrate constitutes the dielectric layer, the outer surface of which constitutes the sensing surface, the sensing lines can detect the wet state of the sensing surface through the dielectric layer by means of electrolytic capacitance, and the detection sensitivity is proportional to the dielectric constant of the dielectric layer and inversely proportional to the thickness of the dielectric layer.

12. (canceled)

13. A system device for monitoring the wet state of a smart absorbent article, comprising a sensor and a smart absorbent article as described in claim 1, wherein the sensor comprises at least two contacts for contacting and electrically connecting the sensing lines in the built-in pocket, and the sensor also comprises a capacitance detecting device for realizing the wetness detection function of the disposable absorbent article by means of electrolytic capacitance.

14. The system device according to claim 13, wherein the sensor comprises at least three contacts, two of which constitute an insertion detection device, when the sensor is inserted into the built-in pocket and the two contacts contact the same sensing line at the same time, the insertion detection device is triggered, and the sensor enters working state.

15. The system device according to claim 13, wherein the sensor also comprises a wireless transmitter and a wireless receiving device for sending and receiving wetness information of the smart absorbent article by wireless means, and the wireless receiving device comprises an audible and visual alarm device, a smart phone, or a personal computer.

16. The smart absorbent article according to claim 4, wherein the sandwich at the front edge of the disposable absorbent article and the back end edge of the disposable absorbent article comprises an invisible opening, each leading to a built-in pocket, wherein the built-in pocket located at the front edge constitutes a functional pocket for inserting a sensor to achieve the wetness detection function, the built-in pocket located at the edge of the back end constitutes a redundant pocket to provide error redundancy for the positioning cut-off during the production of the smart absorbent article.
